(19) United States
(12) Patent Application Publication
(10) Pub. No.: US 2021/0123085 A1
(43) Pub. Date: Apr. 29, 2021

(54) METHOD AND DEVICE FOR THE IDENTIFICATION OF CELL OBJECTS AND TEST COMPOUNDS EFFECTIVE AGAINST THEM

(71) Applicant: MARTIN AND CO. KFT., Budapest (HU)

(72) Inventors: Marton MUHARI, Budapest (HU); László Csaba LENGYEL, Göd (HU); Teréz Mária SVIRZSOVICS-VÁGÓ, Budapest (HU)

(73) Assignee: MARTIN AND CO. KFT., Budapest (HU)

(21) Appl. No.: 16/756,996

(22) PCT Filed: Oct. 17, 2018
(86) PCT No.: PCT/HU2018/050045

§ 371 Date: Apr. 17, 2020

(30) Foreign Application Priority Data
Oct. 17, 2017 (HU) P1700422

(51) Int. Cl. C12Q 1/18; B01L 3/00; H01J 49/00; G01N 1/10; G01N 33/487

(52) U.S. Cl. CPC C12Q 1/18; B01L 3/50273; H01J 49/0036; G01N 21/65; G01N 33/4875; B01L 2400/0475; B01L 2400/0622; G01N 1/10

(57) ABSTRACT

The invention relates to a method for identifying test compounds that have an effect on cell objects, which comprises the following steps: a) providing a liquid sample presumably containing a cell object, b) performing mass spectrometry and/or destructive spectrophotometry testing of the liquid sample according to step a), c) comparing the mass spectrometry spectrum and/or the spectrophotometry spectrum obtained in step b) with the elements of database (s) containing such spectra of known cell objects, d) identifying the cell object present in the sample according to step a) in the course of the comparison according to step c), e) the non-destructive spectrophotometry testing of the sample according to step a), in the course of which the non-destructive spectrophotometry spectrum of the sample is recorded in such a way that test compound is not added to it, and so that test compound is added to it at a given concentration or at several different concentrations, and the recording of the spectrophotometric spectrum of the solution of the test compound, f) comparing the spectrophotometric spectrum measured in the sample without the addition of any test compound and obtained in step e) with the spectrophotometry spectrum of one or more samples prepared with the addition of the test compound, g) drawing a conclusion relating to the effective concentration of the test compound from the result of the comparison according to step f). The invention also relates to a deive serving for implementing the method.

### THE FIELD OF THE INVENTION

The present invention relates to a microfluidic method in the course of the implementation of which the cell objects, especially microorganisms and test compounds that may be used effectively against them, antibiotics in the case of microorganisms, to be found in a sample may be identified with great precision in a short amount of time. The object of the invention also relates to a microfluidic device serving for implementing the method.

### THE STATE OF THE ART

In recent decades the demand for methods with which an infection causing a disease can be identified within a few hours has increased considerably. Furthermore, in recent years the demand for methods capable of identifying bacteria resistance in a short amount of time has grown. The reason for this is that in addition to resistant strains of bacteria, multi-resistant bacteria, i.e. bacteria resistant to several antibiotics, have now become widespread, which can cause more serious, frequently fatal diseases both in humans and animals (especially in livestock). From the data published by the World Bank (see Drug-Resistant Infections—A Threat to Our Economic Future, March 2017, World Bank, http://documents.worldbank.org/curated/en/323311493396993758/p df/114679-REVISED-v2-Drug-Resistant-Infections-Final-Report.pdf) it can be seen that multi-resistant bacteria caused two million human illnesses in the territory of the USA between 2009 and 2011 and among these approximately twenty-five thousand ended in death. At the meeting of the G7 countries in Berlin (Germany) on 8 Oct. 2015 it was stated (https://www.oecd.org/health/health-systems/AMR-Presentation-Kapferer-OECD-Berlin-October2015.pdf) that in the G7 countries one in five human infections is resistant to antibiotics. Although in 2016 a 6% drop could be seen in the preceding seven years in deaths caused by multi-resistant bacteria, it is estimated that in the next 35 years these diseases will cause 300 million deaths worldwide.

The problem of resistant, and especially multi-resistant bacteria, is not only increasingly pressing in human healthcare, but also with respect to livestock. In this respect the struggle against resistant and multi-resistant bacteria starts by establishing the safety of the food chain, and in the course of the overuse of antibiotics these drugs get into the human body with the consumption of the meat of livestock, where they can create resistance (2017. ECDC/EFSA/EMA second joint report on the integrated analysis of the consumption of antimicrobial agents and occurrence of antimicrobial resistance in bacteria from humans and food-producing animals—Joint Interagency Antimicrobial Consumption and Resistance Analysis (JIACRA) Report. EFSA Journal 2017;15(7):4872, 135 pp.).

At present, in certain countries antibiotics were given to animals as a form of prevention, not only in the case of actual infections. This practice is extremely beneficial for the development of resistant and multi-resistant bacteria and puts humans at risk too.

Performing fast screening of livestock on a farm may make it possible to quickly identify individual infected animals and isolate them from the healthy livestock, therefore the entire livestock population can be more effectively protected against the undesirable effects of an infection. So then it is also important to determine the resistance of the microorganism in question to antibiotics on the livestock level, through which a wave of infection spreading over to humans could possibly be avoided.

From the above it can be seen that both in terms of human healthcare and livestock farming it is necessary to provide methods that can help identify the microorganisms causing infections and then the antibiotics that are effective against the microorganisms actually identified and occurring in the sample. Because, as a result of the increasingly probable appearance of resistance and multi-resistance, it is insufficient to rely on the information that may be found in the literature when making a decision on the antibiotic to use against a known bacterium, instead it is preferable to empirically determine the efficacy of a given antibiotic against a given bacterium in each and every case.

One of the first steps in the struggle against a bacterial infection is the identification of the bacterium that is causing the infection. The use of the traditional culturing procedure plays a large role in this. The essence of this process is that a sample presumably containing a bacterium is plated on a suitable culture medium and incubated. Under these conditions the bacteria start to multiply, then after 24 hours or possibly a few days they appear on the culture medium in the form of visible colonies. In the case more precise identification is necessary, samples can be taken from specific colonies for the purpose of growing on a specific culture medium, which can then be spread on a culture medium and incubated once again. As a result of this several separate cultures of the same bacterium will be available. The bacteria colonies developed on the medium can be easily evaluated visually by a person skilled in the art, and the method of evaluation has been dealt with in detail in the literature (see, for example, the International Journal of Food Microbiology, vol. 31, 1-3, August 1996, pp 45-58). The given bacteria colonies can be subjected to biochemical reaction in the interest of the more precise identification of species and serotype, and other molecular methods can be used to evaluate the colonies (e.g. Innovare Journale of Life Science, Vol. 1, Issue 1, Vashist Hemraj, Sharma Diksha, Gupta Avneet: A review on commonly used biochemical tests for bacteria). Another important aspect in addition to determining the species of bacteria is the determination of their amount, for which, when using the culturing method, there are tried and trusted protocols available, a presentation of which may be found in this same publication.

Therefore, the traditional culturing method is an old and thoroughly tested approach, which is still being developed even today. Within the scope of these methods, solutions have been developed with which the reliability of the tests can be increased, such as the use of chromogenic and fluorogenic, and selective and differential culture media (see, for example, Applied and Environmental Microbiology, September 1977, P. 274-279, Edmund M. Powers and Thomas G. Latt U.S. Army Natick Research and Development Command, Natick, Massachusetts 01760 Simplified 48-hour IMVic Test: an Agar Plate Method).

The disadvantage of the culturing method, however, is that it is time-consuming, during which time the infection can spread continuously. The time demand of the culturing method is also increased in the cases of certain strains of bacteria (such as certain strains of Salmonella) by the fact that the culturing step itself must be preceded by an enriching step, which increases the time demand of the entire procedure by a further 12 to 24 hours.

An antibiotic resistance test combined with a culturing process is carried out by adding antibiotic preparations to bacteria colonies created through the culturing process that are probably effective, and by examining which antibiotic preparation actually does kill off the bacterium colony under examination.

The most broadly used method even today for demonstrating antibiotic resistance is the use of resistance discs, which is based on the aforementioned culturing method. The essence of the use of such resistance discs is that a disc containing a given antibiotic is placed on culture medium that visibly contains the bacterium, then it is examined whether the bacteria die in the vicinity of the disc (for example the culture medium that has become opaque due to the presence of the bacteria becomes clear due to the effect of the antibiotic). If the bacteria die in the vicinity of such a disc, this means that the given antibiotic is effective for the given bacterium. The use of resistance discs general requires at least 24 to 48 hours for the first culture to grow then requires a further growing cycle, as in the course of the first growth, first the bacteria found in the samples have to be isolated, then during the second growth cycle new cultures have to be grown from the individual colonies for use with the resistance discs.

If the suitable antibiotic is to be identified with this culturing method, then the time required for this in the case of an internal laboratory test is some 24 to 72 hours (12 to hours of culturing and 12 to 24 hours antibiotic test), while in the case of the use of an external accredited laboratory the amount of time increases by 3 to 6 hours as a consequence of the time required for transporting.

The majority of the bacteria identification, and therefore also bacterial infection identification methods according to the state of the art are based on the traditional culturing method. The main reason for this is that in the initial sample it is difficult to isolate the tissue cell from the bacterial cell, or differentiate between them in the course of testing. Therefore, before identification the number of bacterium cells as compared to the number of tissue cells needs to be increased. Furthermore, in order for measurements of this type to be reliable an amount of bacteria is needed that exceeds the detection limit.

However, the propagation of the bacteria content of a sample is a time-consuming process, and information relating to the nature of the bacteria present (bacterium strain, and the antibiotic that can be effectively used against it) needs to be available as soon as possible in order to effectively prevent the spreading of an infection.

This is why numerous methods have been developed with the help of which the bacteria in a sample can be identified significantly faster than in the culturing process. Numerous solutions may now be found on the diagnostics market for identifying the pathogen in human and animal diseases in just a few hours.

For example, the BioMerieux VITEK 2 device suitable for fast testing is known, which is an automated device capable of quickly identifying various microorganisms (fungi and bacteria) (for details see: http//:www.biomerieux-usa.com/clinical/vitek-2-healthcare). The device uses a database for evaluating the sample and performing identification, the basis of which is the fingerprint of the microorganisms, bacteria serotypes. The fingerprint is provided by the MIC test (Minimal Inhibitory Concentration), i.e. by the pattern obtained on the basis of the result of the dose-effect curve of the antibiotic resistance test related to the entire antibiotic spectrum, which is compared to the patterns stored in the database. The identification of the microorganisms takes place with the help of reagent cards. Each reagent card is a sheet containing 64 different test substrates. The incubation of the sample and the substrate takes place in the reagent card, for which the device provides the appropriate atmosphere and temperature. The reactions to the various substrates are monitored by the device using a transmission optical system. On the basis of the reactions obtained the device is able to identify the given microorganisms with great precision, in a relatively short (3 to 7 hours) amount of time. The disadvantage of the system is that culturing is required in advance, and also it cannot be used in the cases of samples containing various types of microorganism.

International patent application publication number WO/1994/028420 discloses an ELISA (Enzyme Linked Immunosorbent Assay) method for identification of Salmonella, E. coli, Listeria and Staphylococcus bacteria strains in cultures and in foodstuff samples. The method is a direct ELISA method that, following a 24-hour culturing phase, makes it possible to perform a quantitative and qualitative analysis of the above bacteria in the given sample with the help of a luminescence analytical method. Its advantage is that the method is also suitable for identifying other bacteria with the help of suitable bacterium-specific antibodies. Its disadvantage is that it needs having an antibody for each and every bacterium that is to be identified, and an at least 24-hour culturing phase is also required.

International patent application publication number WO/2006/085948 discloses a method based on a PCR (Polymerase Chain Reaction) technique, the essence of which is the identification of the nucleic acid characteristic of the bacterium to be identified. The method makes it possible to multiply (amplify) a small piece of the DNA for the purpose of analysis. The technique is capable of precise bacterium identification, and is capable of handling small sample amounts. Furthermore it is faster (12 to 24 ours) than the traditional culturing procedure, however, its disadvantage is that dead bacteria are also identified, which may give a so-called false positive result, as all DNA present in the sample, even that from the dead bacteria, is identified. A further disadvantage of this method is that it needs a section of the DNA sequence originating from the bacterium to be identified in advance, and also that the process according to the method is slow.

The USA patent publication document number U.S. Pat. No. 5,059,527A discloses a method for identifying the endotoxins of gram-negative bacteria, which is based on the fact that individual bacteria are characterised by their lipid composition. In the course of the method the lipids are obtained from the sample using supercritical extraction, then are transformed into methyl esters of hydroxy fatty acid. The esters obtained are identified using gas chromatography and mass spectrometry. Comparing the obtained lipid distribution with a database makes it possible to identify the bacteria. The advantage of the method is high sensitivity, as it is not necessary to perform culturing of the sample for a long time, and that the method may be automated. Its disadvantage is the special equipment requirement for the supercritical extraction when preparing the sample, and the necessity of performing the chemical reaction.

Patent number U.S. Pat. No. 6,177,266B1 relates to a method for the fast identification of bacteria based on MALDI-TOF-MS (matrix assisted laser desorption ionisation time-of-flight mass spectrometry). The basis of this is a soft ionisation technique, which makes it possible to perform non-destructive testing of proteins characteristic of the given bacterium. MALDI is matrix assisted laser desorption ionisation. This photoionization solution provides well-controllable ionisation which makes it possible to perform the mass spectrometry testing of substances that are very thermally sensitive, such as enzymes, hormones, biomolecules with a molecular mass of several hundred thousand Daltons, proteins, etc. The excitation energy originating from the controllable laser source is absorbed by a matrix and transferred to the molecules to be tested. By appropriately selecting the matrix gentle energy transfer can be achieved with respect to the molecule in the condensed phase. Following this the ions formed are ablated or desorbed from the condensed phase by a large field-energy accelerator system (60 to 100 kV), and then, usually by being linked to a fast analyser (TOF), the large molecular mass (as many as 105 to 106 Dalton) ions can be measured, this combination is the MALDI-TOF. In the course of the measurement the given microorganism can be identified on the basis of the mass profile (fingerprint) derived from the sample. Today the MALDI-TOF is an analytical measurement system that is capable of identifying the presence of compounds present at the lowest amount, and it can even identify material from a matrix at a concentration of ˜10⁷/ml. Its disadvantage is that it is better at handling samples originating from a pure bacteria culture. Although this method makes bacteria identification possible that is faster and more precise than previous methods, it is still not viewed as being sufficiently fast, because the 24- to 72-hour culturing period cannot be avoided. One of the disadvantages of the MALDI-TOF approach is that it is exceptionally expensive.

As mentioned above, the identification of the bacteria is, in itself, insufficient, as due to any possibly existing resistance or multi-resistance it is also necessary to identify the antibiotic that can be used against the bacteria that are actually identified in the interest of planning effective treatment.

There are devices available commercially, such as the aforementioned BioMérieux VITEK 2 device, or the device manufactured by Thermofisher with catalogue number R8311002 (see: https://www.thermofisher.com/order/catalog/product/R8311002), which are used in the course of human clinical studies, and these can be used to determine the antibiotic resistance specifically with respect to a certain bacterium. These instruments, however, are expensive and their applicability is limited. Because culturing usually has to be performed before measurement in the course of the procedures that can be initiated with these instruments. Exceptions to this are tests based on enzyme reactions, where the user is only expecting a colour reaction. (see, for example, https://www.thermofisher.com/hu/en/home/industrial/microbiolog y/microbial-identification/biochemical-identification/biochemical-id-panels.html). Furthermore, the applicability of these methods is split up into anaerobic and aerobic bacteria, and into different groups of bacterium types, i.e. different types of device are needed to test different groups of bacteria. The disadvantage of the method using an enzyme reaction, without culturing is that substrates specific to the given enzyme reaction are required, and it is necessary to input further reagents into the system in order to detect the colour reaction.

Quick tests are also known of that are suitable for quickly identifying a given bacterium beside the patient's bed. Usually, these tests are based on a colour reaction and provide a yes or no type response and their applicability also covers a narrow bacteria spectrum, and can only be used for identifying a small number of bacterium strains.

Using the presently available methods a bacterial infection in the body of a mammal, either an animal or human body, may be tested if the bacterium causing the infection starts to predominate after balance in the body is lost and proliferates, gets into the blood circulation, and from there into the various organs, where it appears causing a pathological change. When using the testing methods based on the traditional culturing process a minimum of 48 or even 72 hours may be required, sometimes even many times this amount of time, in order to identify the infecting bacterium and determine the antibiotic resistance. Although the treatment of a disease caused by a bacterial infection should not be started until the bacterium behind the infection and its antibiotic resistance have been determined, in practice treatment is still started at the time the symptoms appear on the basis of the clinical condition, the physical changes that can be visually detected, and general experience, in other words without knowledge of the bacterium or of its antibiotic resistance. This is done in the interest of preventing the consequences of the infection in good time, as during the time required for the laboratory tests lasting several days the spreading of the infection can cause significant damage, on a livestock farm, for example. In practice the consequence of this may be that the doctor misdiagnoses the disease and the antibiotic used is unsuited for treating the pathogen present in the body. Another, serious consequence of this practice is that it allows any pathogen or bacterium not yet producing a pathological condition to develop antibiotic resistance. Furthermore, it frequently occurs in the case of baseless, fast diagnoses that the antibiotic therapy administered is not effective due to the existing resistance, and so with this time is lost, proliferation of the bacteria is facilitated, the condition and physical condition of the patient, irrespective of whether it is a human or an animal, considerably deteriorates and the spreading of the infection is not stopped.

The inventors of the present invention developed a method in the past with which the bacteria present in a sample may be identified with great precision. The starting point was that the main constituent components of every cell membrane are the phospholipids, the ratio of which is characteristic of a given bacterium. The essence of the method was that a mass spectrometry (MS) image was made of a sample presumed to contain the bacterium to be identified. This method involved performing measurement in the 50 to 2000, preferably within this, in the 600 to 900 mass to charge range suitable for identifying phospholipids. With this an MS spectrum is obtained containing a phospholipid pattern characteristic of the given bacterium. If the MS spectrum obtained in this way is compared with the elements of a database the elements of which contain the MS spectra of known bacteria, then the bacterium found in the tested sample may be identified as long as the database used contains such an MS spectrum.

The mass spectrometry method developed by the inventors of the present invention in the past and summarised above is preferable to the method described in the aforementioned patent number U.S. Pat. No. 6,177,266B1. As in the course of the method disclosed in patent number U.S. Pat. No. 6,177,266B1 the so-called MALDI-TOF-MS procedure is used to map out the protein content of bacteria, which procedure is necessarily preceded by pre-incubation, culturing, then another 7 to 14 hours before the desired spectrum is obtained. In a sample that has not been cultured, as a consequence of the use of the TOF module the procedure is so much more sensitive that the high noise level is disturbing. In the course of the method developed by the inventors of the present invention the phospholipid concentration of bacteria is mapped out, which does not require preliminary incubation and the desired spectrum can be obtained in about 3 hours.

Furthermore the MALDI-TOF-MS process requires a matrix, and also during this process the proteins may not be fragmented. In the method used by the inventors of the present invention there is no need for a matrix, and the phospholipid molecules may be fragmented. As known internal standard bacteria have to be mixed with the sample when using the MALDI-TOF-MS process, which is not required in the MS method used by us. The reason for this is that MALDI is a soft ionisation technique using a relatively low voltage, whereas in the case of the simple MS used by us the voltage may be varied, and by increasing the voltage the phospholipid molecules start to fragment, i.e. variable ionisation is used. In this case the variable ionisation approach is required because with the optimal determination of the degree of ionisation it becomes possible to identify the phospholipid molecules as well as the quality of the apolar and polar components appearing as their fragments. The amount of data is multiplied many times as a result of the fragmentation. Comparing all of this, a more detailed MS spectrum of the bacteria may be obtained using the faster, simpler and cheaper MS method developed by us previously than with the method described in patent number U.S. Pat. No. 6,177,266B1.

On the basis of the above then there is a need for a method that may be performed quickly that supports a consultant physician/veterinary surgeon when making a selection decision in connection with treatment and for an easily accessible device that is able to implement such a method, which method is able to identify the microorganisms occurring in a sample with high reliability and determine the antibiotic and its approximate dose that is able to kill such an identified microorganism. On the basis of this the decision on the necessary steps may be made quickly, primarily the decision on which antibiotic may be used to effectively treat an infection that has occurred and on the dose the antibiotic needs to be administered at.

### BRIEF DESCRIPTION OF THE INVENTION

We recognised that if a mass spectrometry database of the phospholipid patterns of microorganisms causing infectious diseases and/or a spectrophotometry database of microorganisms causing infectious diseases are prepared and a sample presumably containing microorganisms is subjected to mass spectrometry and spectrophotometric testing, and the spectrum obtained as a result of the test is compared with the spectra contained in the database, then the microorganisms occurring in the tested sample may be properly identified, furthermore after adding presumably effective antibiotic solutions at various concentrations to the sample and subjecting it to spectrophotometric testing, then the antibiotic effective against the identified microorganisms and its effective dose may be determined in a short amount of time.

It was also recognised that as the cell membrane of every cell object contains various phospholipids in proportions characteristic of the given life form, then the invention is not only suitable for identifying microorganisms, but also for identifying any cell object and the compounds that are able to significantly influence, or, in practice, destroy this cell object.

In accordance with the above objective and recognition, the task was solved with a method serving for identifying test compounds that have an effect on cell objects, which method comprises the following steps:

a) providing a liquid sample presumably containing a cell object,

b) performing the mass spectrometry and/or destructive spectrophotometry testing of the liquid sample according to step a),

c) comparing the mass spectrometry spectrum and/or the spectrophotometry spectrum obtained in step b) with the elements of database(s) containing such spectra of known cell objects,

d) identifying the cell object present in the sample according to step a) in the course of the comparison according to step c),

e) the non-destructive spectrophotometry testing of the sample according to step a), in the course of which the non-destructive spectrophotometry spectrum of the sample is recorded in such a way that test compound is not added to it, and so that test compound is added to it at a given concentration or at several different concentrations, and then the recording of the spectrophotometric spectrum of the solution of the test compound,

f) comparing the spectrophotometric spectrum measured in the sample without the addition of any test compound and obtained in step e) with the spectrophotometry spectrum of one or more samples prepared with the addition of the test compound obtained in step e),

g) drawing a conclusion relating to the effective concentration of the test compound from the result of the comparison according to step f).

According to a preferable form of implementation of the method according to the invention, both the mass spectrometry and the destructive spectrophotometry test are performed in the course of step b) and the comparison according to step c) is performed with respect to both the mass spectrometry and the spectrophotometry spectra.

According to another preferable form of implementation of the method according to the invention the mass spectrometry test according to step b) is performed in the 50 to 2000 mass to charge range, preferably in the 600 to 900 mass to charge range.

According to another preferable form of implementation of the method according to the invention a UV spectrophotometry or Raman spectroscopy test is used as the spectrophotometry test during the implementation of step b) and/or step e).

In the course of another preferable form of implementation of the method according to the invention, in the case of the destructive spectrophotometry test according to step b) the destruction is performed with ultrasound or electromagnetic radiation, preferably laser light, even more preferably using laser light at a wavelength of approximately 530 nm.

According to another preferable form of implementation of the method according to the invention the cell object is a microorganism, preferably a bacterium, especially preferably a bacterium causing a human or animal disease, and the test compound in the case of this especially preferable form of implementation of the method is an antibiotic.

According to another preferable form of implementation of the method according to the invention the elements of the databases according to step c) are spectra that were recorded using the same instrument as used when performing the tests according to step b).

According to another preferable form of implementation of the method according to the invention the comparison according to step c) and/or the identification according to step d) and/or the comparison according to step f) and/or the drawing of the conclusion according to step g) are performed using a computer algorithm.

According to another preferable form of implementation of the method according to the invention the sample is a piece of the tissue/part of an animal or human probably affected by an infection, a mucosal smear, a sample originating from body fluid, or a sample of epidermis. In this respect an animal is understood to mean preferabyl livestock animals, especially pigs, cattle, sheep, horses, oxen, goats, poultry, fish, turkeys, geese, pigeons, ducks, ostriches.

The method according to the invention is preferably implemented in a microfluidic device.

According to the above objective and recognition the task was solved with a device serving for identifying test compounds having an effect on cell objects that is a microfluidic device and contains

a sample holder 1, a first pump 11, a first distribution valve 12, and a mass spectrometer 13,

one or more test compound holders 2a, 2b, 2c, and a second pump 21,

an oil container 3, and a third pump 31,

a drop dispenser 4, a microfluidic tube 5 containing a first window 52a and a physically identical or physically different second window 52b, a destruction element 6, a spectrophotometer 7, a second distribution valve 51 and a collector 8

wherein

a first pump 11 transports a part of the liquid sample from the sample holder 1 through a first distribution valve 12 into either the mass spectrometer 13 or a drop dispenser 4, and a second pump 21 transports the given test compound solution from the one or more test compound holders 2a, 2b, 2c into a drop dispenser 4,

a third pump 31 transports oil from the oil container 3 into the drop dispenser 4, and

the drop dispenser 4 dispenses a part of the liquid sample transported to it via the first pump 11 and the first distribution valve 12, to which it optionally mixes test compound solution transported to it via the second pump 21, and alternately the oil transported to it through the third pump 31 into the microfluidic tube 5, through which the liquid sample parts and the oil drops alternately flow, and where in case A) the destruction element 6 exerts a destruction effect on the one or more liquid sample parts passing in front of the first window 52a, and the spectra of the one or more liquid sample parts passing in front of the second window 52b are recorded using the spectrophotometer 7, and then the one or more liquid sample parts are transported to the collector 8 through the second distribution valve 51, or

in case B) one or more liquid sample parts flowing from the drop dispenser 4 pass in front of the second window 52b without destruction and the spectra of the one or more liquid sample parts are recorded with the spectrometer 7, which liquid sample parts are then transported to the collector 8 through the second distribution valve 51, or are returned into the drop dispenser 4 through the second pump 21, in the course of which test compound solution is added to the given liquid sample part from the appropriate test compound holder 2a, 2b, 2c through the second pump 21 thereby increasing its test compound concentration, and in this way the changed liquid sample part once again passes in front of the second window 52b through the microfluidic tube 5 without destruction and the spectrum of this liquid sample part is recorded with the spectrometer 7, then this is either transported into the collector 8, or in accordance with the former process the test compound concentration of the tested liquid sample part is increased even more, and the spectrum of the test compound transported from the appropriate test compound holder 2a, 2b, 2c through the second pump 21 and the drop dispenser 4 into the microfluidic tube 5 is recorded with the spectrometer 7.

According to another preferable embodiment of the invention the first window 52a and the second window 52b of the microfluidic tube 5 are physically the same.

According to a preferable embodiment of the invention the spectrometer 7 is a UV spectrophotometer or a Raman spectrometer, preferably a Raman spectrometer.

According to yet another preferable embodiment of the invention the spectrometer 7 is a Raman spectrometer and this same Raman spectrometer also serves as the destruction element 6 as a result of the laser light emitted by it.

According to yet another preferable embodiment of the invention the destruction element 6 is an element emitting electromagnetic radiation, preferably an element emitting laser light, even more preferably an element emitting laser light at a wavelength of approximately 530 nm.

According to yet another preferable embodiment of the invention the destruction element 6 is an element emitting ultrasound.

According to a preferable embodiment the temperature of the microfluidic device according to the invention may be controlled.

According to a preferable embodiment of the microfluidic device according to the invention an inert atmosphere may be created in it.

### IN THE FIGURES

FIG. 1 shows a schematic view of the operation of the microfluidic device according to the invention,

FIG. 2a shows the MS spectrum of Campylobacter jejuni present in a sample originating from animal gastrointestinal tissue,

FIG. 2b shows the MS spectrum of Clostridium perfringens present in a sample originating from foodstuff,

FIG. 2c shows the MS spectrum of Escherichia coli present in a sample originating from human tissue,

FIG. 2d shows the MS spectrum of Staphylococcus auerus present in a sample originating from animal leg joint tissue,

FIG. 3a shows the Raman spectrum of Escherichia coli originating from a pure culture irradiated with 100% intensity, 785 nm laser light,

FIG. 3b shows the Raman spectrum of Escherichia coli originating from a pure culture irradiated with 100% intensity, 785 nm laser light, following treatment for 20 minutes with sulphonamide antibiotic,

FIG. 3c shows the Raman spectrum of Escherichia coli originating from a pure culture irradiated with 100% intensity, 785 nm laser light, following treatment for 30 minutes with sulphonamide antibiotic.

### DETAILED DESCRIPTION OF THE INVENTION

In the terms of the present invention microorganisms are understood to mean microscopic living beings, i.e. that are not visible to the unaided eye. These may be bacteria, fungi, archaea and protists. With respect to that viruses and prions are not viewed as living beings, they are not understood as being included within the concept of microorganism.

In the terms of the present invention overall the microorganisms according to the above and objects that are not viewed as microorganisms but consist of one or a maximum of a small number of living cells are called cell objects and that, as a result of their size, can be handled in a microfluidic device (see below). In addition to microorganisms these also include, but are not limited to, plant, animal and human cells, algae, fungi, cells taken from cell cultures, blood cells, chimeras, stem cells, and artificial cells.

By using the method according to the present invention one or even several types of cell object may be identified in a sample. If the expression cell object is used in the singular in the specification, unless some other meaning can be derived from the context, this is not understood to mean just one type of cell object.

In the terms of the present invention sample is understood to mean a biological sample that presumably contains a cell object, preferably a microorganism that needs to be identified, for example in the interest of fighting against or preventing the spreading of an infection. The sample is typically but not necessarily a probably infected piece of tissue/part of an infected animal or human, a mucosal smear, a sample of body fluid, a sample of epidermis, or any material originating from the examined body. In this respect animal is preferably livestock, especially pigs, cattle, sheep, horses, oxen, goats, poultry, fish, turkeys, geese, pigeons, ducks, ostriches. Accordingly the sample may be for example but not limited to: a biopsy taken from the internal organs of a living individual, a sample taken from the internal organs of deceased animals or persons (such organs, for example, include the organs constituting the gastrointestinal system, the organs constituting the respiratory system, the organs constituting the blood circulation system, organs constituting the nervous system, the organs of the skeleton and musculature, the organs of the excretory system, as well as the reproductive organs and the glands), the sample may also be smears taken from the mouth, nose and throat cavities, smears taken from the mucosa of the genitals, urinary tract, and rectum, it may also be a sample taken from the skin, hair, fur, feathers, scales, nails, claws, auditory canal, anus or their vicinity, a sample taken from the genitals or their vicinity, from the cloaca or its vicinity, the eyes or their vicinity, from the mouthpart or its vicinity, it may also be a sample taken from blood, urine, seminal fluid, vaginal discharge, nasal discharge, tears, sweat, stool, eggs, and in the case of a new-born human/mammal the placenta, the umbilical cord, and foetal membrane may also serve as the source of the sample. As stated above the sample does not necessarily have to originate from a human or animal, it may be some other kind of sample that may contain an identifiable microorganism. Examples of such a sample may be, but are not limited to cell culture, a piece of soil, a piece of clothing textile, a sample originating from clothing, a hospital instrument/object, a tool/object used in livestock farming, any waste, a sample of food, foodstuff raw material, an instrument/object used for eating, feed, medicines, packaging material, a sample originating from a surface (such as a building, means of transport, production facility), material evidence from a crime scene, etc.

In the terms of the present invention test compound is understood to mean all compounds that are tested with the method according to the invention to determine whether they have any effect on a cell object. Test compounds are typically viewed as being antibiotics in the course of testing microorganisms, especially bacteria. In this respect an effect is understood to mean when the given test compound changes a significant characteristic of the given cell object. In terms of the present invention this effect is examined using non-destructive spectrophotometry testing, such as with Raman spectroscopy, in other words it is determined whether a given test compound at a given concentration causes any changes to take place in the spectrophotometry spectrum of the cell object. Consequentially, in the terms of the present invention, changes that cannot be demonstrated with the spectrophotometry used are not deemed to be effects. It is the effect of antibiotics as test compounds exerted on bacteria as cell objects that are especially preferably tested for with the method according to the invention, where the effect consists of the bacterium being destroyed.

In the terms of the present invention antibiotics are understood to mean all test compounds that are capable of destroying microorganisms. In the terms of the present invention when reference is made to an antibiotic that is effective against the identified microorganism, this is not understood as meaning the antibiotic that is usually effective against the identified strain of microorganism, instead an antibiotic that is effective against representative individuals of the microorganism that may be found in the tested sample. Nevertheless, as is obvious for a person skilled in the art, an antibiotic identified in this way is, naturally, very probably effective against other representative individuals of the identified microorganism strain also.

Those low-pressure (vacuum, atmospheric pressure) or high-pressure (such as several hundred or thousand bar) systems are called microfluidic devices that are suitable for continuously or intermittently transporting liquids, sludge, suspension, gas, steam in one or several phases, and for manipulating their flow. The system may contain a dispenser, nozzle, pump, needle, valve, filter, mixer, reactor, separator, distributor, as well as components that are capable of actively or passively, directly or indirectly causing chemical and biological reactions to occur and capable of performing analysis. It is not essential but optionally the system should have a mechanical or electronic regulation, control and assessment unit. The components are connected together by a tube network, where the diameter of the tubes does not exceed approximately 1.0 mm, and the surface area to volume ratio is favourable for the creation of laminar flow of liquids and high-pressure, liquid-state gases.

Pico- and nano-fluidic devices differ from microfluidic devices in that the diameter of their tubes does not exceed approximately 1 μm.

Meso-fluidic devices differ from microfluidic devices in that the diameter of their tubes exceeds approximately 1.0 mm, and the surface area to volume ratio does not necessarily facilitate the development of laminar flow, these devices being ideal for handling turbulent flow single or multiphase systems.

In accordance with the above the present invention relates to a method with which test compounds having an effect on cell objects can be identified. Here identification is not only understood to mean which test compound has an effect that can be identified using spectrophotometry on the given cell object, but also the concentration at which the test compound exerts the effect in question.

As step a) of the method according to the present invention, a liquid sample is provided presumably containing the cell object in accordance with the above.

Before performing the test, if it is not liquid the sample must be taken into liquid state before the test is performed in order for it to be tested in the microfluidic device. The essence of this is that the sample to be tested presumably containing a cell object is placed in a buffer that does not damage this cell object, or at least does not change its characteristics to be tested with the test compound. For example, when testing the effect of antibiotics exerted on bacteria it must be taken into consideration that the bacteria should not be destroyed in the liquid sample. It is obvious for a person skilled in the art that the type of liquid medium in which a characteristic to be tested for is maintained is itself a characteristic of the cell object, in other words the measures to be implemented in the course of step a) belong to the mandatory knowledge of a person skilled in the art. The placing of the sample containing the cell object into such a buffer, i.e. sample preparation, may take place by dissolving, washing, soaking, diluting, etc.

It is obvious for a person skilled in the art that the liquid sample to be tested may already be available as a result of the nature of the sample, therefore no further measures have to be performed in order to implement step a). Such samples include samples taken from wastewater or drinking water. Naturally, such cases are viewed as the implementation of step a).

It should be noted that in connection with the sample to be tested with the method according to the present invention it can only be stated that it only presumably contains the cell object to be tested, especially if the purpose of the test is to determine whether the sample contains this cell object at all. If the sample does not contain such a cell object, then this will be determined in the course of the implementation of step d) of the method and then there will be no reason for continuing the method.

According to the above the mass spectrometry and/or destructive spectrophotometry testing of the liquid sample according to step a) is performed as step b) of the method according to the present invention. Therefore, according to the invention, either the one or the other of the two processes, mass spectrometry or spectrophotometry, or both of them are used. The reason for this is that this step provides the data for the next steps c) and d), in other words the objective is to identify the cell object in the tested sample. Both mass spectrometry and spectrophotometry are testing methods that are well known to a person skilled in the art.

This identification may take place using a mass spectrometry test or with a destructive spectrophotometry test. According to a preferable embodiment of the present invention both spectroscopy methods are use, as by using two different processes for the same sample the result obtained is significantly more precise, as in this way it is very probable that the errors originating from the unique features of the individual processes can be overcome.

Essentially what takes place during mass spectroscopy testing is that the sample brought to the liquid state is placed in the MS device either with or without performing chemical destruction, where depending on the settings it decomposes, becomes ionised and the phospholipids forming the cell wall become fragmented, and after determining the masses of which the fingerprint of the cell object is obtained.

Essentially the procedure performed in the course of destructive spectrophotometry testing is that the sample is handled in such a way that the cell objects it presumably contains are destroyed. The spectrophotometric spectrum of a solution obtained in this way is characteristic of a given cell object, in other words this provides the fingerprint of the given cell object.

The mass spectrometry test according to step b) is preferably performed in the mass to charge range of 50 to 2000 or even more preferably in the mass to charge range of 600 to 900. As this mass to charge range is suitable for performing the MS identification of the phospholipids forming the cell membranes. In other words a test performed in this range provides a suitably detailed phospholipid fingerprint of the tested cell object. The amount of ionisation may be varied in the course of the MS testing, therefore, on the one part, it is possible to identify larger sized phospholipid molecules, and, on the other part, the smaller apolar and polar fragments can also be identified. The two most common phospholipids, phosphatidylethanolamine (PE) and phosphatidylglycerol (PG) are typical constituents of the spectra.

The destruction performed in the course of the destructive spectrophotometry testing according to step b) is understood to mean the destruction of the cell object in the tested sample in the interest of the recorded spectrum being detailed with cell components from within the cell. The destruction may take place with a device known from the prior art suitable for destructive testing, such as with electromagnetic radiation (e.g. laser light), enzyme destruction, ultrasound destruction, thermal effect destruction, radiation destruction, and destruction performed with a chemical (e.g. with acids, alkalis, high ion strength solutions, distilled water, etc.).

The destruction according to step b) is preferably performed using ultrasound or electromagnetic radiation, preferably with the use of laser light, even more preferably with the use of laser light at a wavelength of approximately 530 nm. Most preferably in the case of the use of a Raman spectrometer as the spectrophotometric device the destruction takes place using the high-energy laser light emitted using this device. The Raman spectrum itself can then be recorded immediately after such destruction using lower energy laser light. It is obvious for a person skilled in the art that the smaller the wavelength of electromagnetic radiation (e.g. laser light), the greater its energy. According to our experience laser light at a wavelength of 530 nm is suitable for the destruction of cell objects. Naturally other electromagnetic radiation with a wavelength in a lower category is also suitable for this purpose and it is also obvious that by increasing the wavelength the destructive effect is reduced, however, the limit of effectiveness becomes obscure. The energy of the electromagnetic radiation may also be influenced in the range between 0 and 100% by changing the intensity. As will be explained later on, lower energy electromagnetic radiation is used for the non-destructive spectrophotometry testing according to step e). During our experiments it was found that laser light at a wavelength of 785 nm is especially suitable with the Raman spectroscopy process. If only shorter wavelength electromagnetic radiation is available for the non-destructive spectrophotometry testing, then the greater energy resulting from the shorter wavelength may be reduced to a lower level by reducing the intensity. In summary then suitably higher or lower energy electromagnetic radiation is to be used for the destructive or the non-destructive spectrophotometry testing, where this energy may be controlled both with the wavelength and with the intensity used. Nevertheless, approximately 530 nm wavelength laser light is especially preferable in the case of destruction performed with laser light, as this destruction process may be easily automated, is fast, does not require any chemicals to be added, and produced good results in the case of our experiments.

As a result of the implementation of step b) MS and spectrophotometry spectra are obtained on the cell objects in the tested sample, which spectra are characteristic of the given cell object.

In the course of the implementation of steps b) and/or e) UV spectrophotometry or Raman spectroscopy testing is preferably used as the spectrophotometry test, most preferably Raman spectroscopy testing is used. The advantage of these two spectrophotometry testing methods is that, taking into consideration the objectives of the method according to the present invention, they have a suitable level of precision, may be performed quickly and are easy to build into a microfluidic device. Both spectroscopy methods proved to be suitable for the application in the course of our experiments. A further advantage of the Raman spectroscopy approach is that the laser light used in this case may be tuned so that it is used in the course of the destruction step, and also used to perform the non-destructive spectrum recording. The use of Raman spectrometry is also preferable because the presence of water molecules does not disturb measurement with consideration to the fact that the tested sample is typically available in an aqueous medium.

In step c) of the method according to the present invention, according to the above the mass spectrometry spectrum and/or the spectrophotometry spectrum obtained in step b) is compared with the elements of database(s) containing such spectra of known cell objects. This comparison is essentially the subtraction of the measured spectrum and of the corresponding spectrum in the database from each other. This step assumes the existence of the aforementioned databases. These databases therefore contain MS or spectrophotometry spectra as database elements, which spectra were previously recorded of known cell objects, such as bacteria. In other words each element in such a database contains a spectrum and the name of its corresponding cell object or other identifier. This means that such a database contains spectra about each of which it is known which cell objects, such as bacteria, they correspond to. These databases may be available in digital format, e.g. on a computer or on a remote-access computer (e.g. in the cloud), on digital data carrier or even on a physical carrier, such as on paper. According to the invention spectra available in digital format are preferable.

It is obvious for a person skilled in the art that several databases may be used in the course of step c), either several MS databases, or several spectrophotometry databases, or several databases of both types. It is also obvious that the MS and spectrophotometry databases do not necessarily contain spectra relating to the same cell objects, in other words they do not necessarily relate to the same cell objects. Nevertheless it is preferable if the MS and spectrophotometry databases used relate to the same cell objects, because this ensures greater precision for the identification of the cell objects.

The spectra recorded with the method according to the present invention may be compared with the spectra according to the aforementioned databases in a known way, for example by visual inspection or using a computer algorithm. Obviously the spectra recorded with the MS spectroscope are compared with the elements of a database containing MS spectra, and the spectra recorded with the spectrophotometry spectroscope are compared with the elements of a database containing such spectra. The methods serving for comparing both MS and spectrophotometry spectra are known of to a person skilled in the art.

The comparison is performed by jointly carrying out main component analysis and discriminant analysis. The basis of this is that the known microorganisms recorded in the database are placed into groups, then the sample is placed into one of the groups on the basis of its spectrum. If the sample cannot be reliably categorised into any group, then it is placed into a group of unknown spectra. Intensity value pairs were established in the examined mass to charge range on the basis of mass number, then these were placed into intervals and it was examined which groups the fingerprint of the sample correlates with. If a sample contains several types of cell object, even then the mass spectra obtained as a result of the main component and discriminant analysis used make it possible to clearly identify the cell objects in the tested sample, in such cases the mass spectra of the cell objects individually identified from the database and the mass ranges determined as other noise are subtracted from each other, as a consequence of which the data obtained in this way make it possible to isolate the various cell objects found in the sample mixture from each other.

Although it is obvious for a person skilled in the art, in the terms of the present invention comparison means the comparison of spectra recorded using identical methods, in other words an MS spectrum is compared to an MS spectrum, and although the spectrophotometry method is mentioned in itself, obviously a UV spectrophotometry spectrum can only be compared to another UV spectrophotometry spectrum, and similarly a Raman spectrum can only be compared to a Raman spectrum. What is more, there may be other parameters that need to be identical in order for spectra to be comparable.

The result of the comparison is that it is determined whether the databases contain spectra similar to the spectra recorded in the course of the method according to the present invention.

According to a general principle in connection with the performance of measurements that is known by all persons skilled in the art the more similar the measurement conditions the more comparable the results of two or more measurements are, and it is best if the measurements are performed under identical conditions with an identical type of device. This general principle is also true for the MS and spectrophotometry measurements performed according to the present invention. As explained above, the MS and spectrophotometry spectra measured during the implementation of the present invention are compared to the MS and spectrophotometry spectra of a database created in advance, and it is examined whether there are any in the database that are so similar that make it possible to identify the cell object present in the sample, furthermore the effect of a test compound on the cell object in the given sample is examined, in the course of which examination the spectrophotometry spectrum recorded of a sample without the test compound is compared to the spectrophotometry spectra of samples containing the test compound at various concentrations. Naturally spectra recorded using different devices and under different condition can also be compared, but the more similar the conditions are the more precise the result of the comparison is. In other words if the spectra are recorded with the same type of device a more precise result is obtained than when comparing spectra recorded with different types of device.

If during step c) one or more spectra in the MS spectra database are found to be similar to the MS spectrum recorded during the MS testing, or one or more spectra in the Raman spectra database are found to be similar to the spectrophotometry spectrum recorded during the spectrophotometry testing, then in the course of step d) it can be determined from the database(s) what the one or more cell objects, for example bacteria, are that have a spectrum/spectra similar to the spectrum recorded of the sample according to step a). In this way, therefore the cell object(s) contained in the tested sample can be identified. From the above it can be seen that using the method according to the present invention not only can the identity of one cell object be determined from a given sample but of several cell objects as well.

According to the present invention both MS spectroscopy and spectrophotometry can be used for the identification of cell objects, or even both processes can be used for the same sample. Determining which process to choose depends on which device is available and on what databases are accessible for the comparison according to step c). In the terms of the present invention it is preferable if a sample is examined using both processes, as in this way at least some of the uncertainties resulting from the unique features of the individual processes can be ruled out and the cell objects present in the sample identified with greater precision.

If during the implementation of step d) no cell objects are identified in the sample according to step a), then the conclusion may be drawn that the tested sample does not contain a cell object that has its spectrum in the databases according to step c). This does not mean that the sample according to step a) does not contain any cell object for certain. For example, if MS and spectrophotometry spectra databases are used that contain spectra recorded about the same five bacteria, then if no bacteria are identified in one sample in the course of the implementation of step d), that means that the sample does not contain any of the bacteria contained in the database, however, the presence of other bacteria and/or microorganisms cannot be excluded on the basis of this.

After the cell objects in the sample according to step a) have been identified in step d), the following step e) includes the testing of the test compounds that presumably have an effect on this cell object. For example, if the investigation is directed at determining the types of antibiotic and the doses with which bacteria in a sample can be destroyed, then the one or more antibiotics are used as the test compounds that are effective against the bacterium identified in step d) according to the literature.

It is important to perform a non-destructive spectrophotometry test in the course of step e), in other words the liquid sample according to step a) is placed in the spectrophotometer without implementing a destruction step, meaning that intact cells are examined. In the case of step e) electromagnetic radiation of a wavelength and intensity, such as laser light, must be used for the non-destructive spectrophotometry measurement that is of sufficiently low energy to not damage the cell objects that are presumably in the sample. In the case of Raman spectroscopy laser light at a wavelength of 785 nm has proven to be suitable for this purpose.

According to step e) first of all a spectrophotometry spectrum is recorded of a sample to which no test compound is added. The spectrum obtained in this way serves as a reference, in other words the changes occurring as compared to this spectrum are examined. Also the spectrophotometry spectra of samples are recorded, preferably but not necessarily under the same measurement conditions, to which the test compounds to be tested have been added in given concentrations. In addition the spectrum of the solution of the test compound is recorded, with which the spectra of the liquid sample parts containing the test compound can be corrected.

Determining the concentrations of a test compound that are worth testing with respect to a given cell object belongs to the obligatory knowledge of a person skilled in the art, as does the amount of time a given liquid sample part needs to be incubated with the test compound. Because, on the one part, a person skilled in the art may know the concentrations of test compounds that are effective on the individual cell objects from the literature, for example it is known in general what doses antibiotics distributed for medicinal purposes can be used at against a given bacterium, from which the concentration required for the testing according to the present invention can be calculated. This is a good initial basis for determining the concentrations that are worthwhile trying out in the course of the method according to the present invention. If the probably effective concentration of a test compound is not known, then obviously it worth starting the test using wide concentration ranges and then restricting the range in the light of the results. Similarly a person skilled in the art may easily determine the incubation period on the basis of data from the literature. For example, in the case of testing antibiotics effective against bacteria an incubation period of about 20 minutes is usually sufficient.

In the course of the method according to the present invention it is possible to test the test compound even at just a single concentration if the question being asked is whether a test compound is effective at a given concentration against a given cell object. In other words a test involving a single measurement point also belongs to the scope of protection of the present invention. Theoretically the number of measurement points has no upper limit, this is determined by the measurement arrangement, the amount of sample and test compound available, and the amount of time available for performing the test, which parameters change from case to case.

If the spectra recorded in the course of step e) are available, i.e. spectrophotometry spectra of the sample without the test compound and of the sample containing the test compound at various concentrations, and the spectrum of the solution of the test compound itself, then in step f) the reference spectrum (i.e. the spectrum recorded of a sample not containing the test compound) and the spectra recorded of samples containing the test compounds at various concentrations are compared, and then this comparison is corrected using the spectrum of the solution of the test compound. In the course of making the comparisons it may be determined whether a test compound has caused any change in the cell object (e.g. bacterium lysis), as a consequence of which its spectrophotometry spectrum changes. The change may be caused, for example, by the lysis of the cells of the tested cell object as a result of the effect of the test compound, or, for example, the metabolism of the cells may change due to its effect and so the change in the metabolism products may be observed in the spectra.

In step g) the conclusions are drawn from the result of the comparisons according to step f). If the test compound is used at a concentration lower than what is effective, then no significant change can be expected in the spectrum, if the test compound is used at an effective or higher concentration, then a significant change can be expected in the course of making the comparison. From this result a person skilled in the can clearly conclude the concentration value at which the effective concentration of the test compound starts.

If made necessary by the purpose of the testing, it may be worthwhile performing the method according to the invention using several concentration values in a narrower concentration range in the vicinity of the concentration value that is barely effective, thereby making the result relating to effective concentration more precise.

Therefore, the method according to the present invention relates to the identification of cell objects and to the determination of the effective concentration of test compounds that are effective with respect to these cell compounds. In practice, first of all the microorganisms, most frequently bacteria that cause an infection are required for such a method. As there is a frequent need for the identification of the pathogen that causes a bacterial human or animal infection and the type and effective concentration of an antibiotic that is effective against it, and in such cases the amount of time required to determine this information is also a significant factor. The method according to the present invention is especially suitable for investigating infections on livestock farms, for example, or for use in human healthcare institutions, which infections are usually caused by microorganisms, and most frequently by bacteria. As with the implementation of the present method it can be determined within a few hours what type and dose of antibiotic needs to be used to treat an infection occurring on a poultry farm, for example, so preventing significant mortalities. It is also obvious for a person skilled in the art why in the case of a human infection it is exceptionally important to determine the type of microorganism that is causing it as quickly as possible, i.e. preferably the type of bacteria and the type and dose of the antibiotic that may be used against it.

With respect to that the method according to the present invention may be preferably used for the identification of microorganisms, even more preferably for the identification of bacteria, in harmony with this the sample tested with the method according to the present invention is preferably a piece of the tissue/part of an animal or human probably affected by infection, such as a mucosal smear, a sample originating from body fluid, or a sample of epidermis. As it is these sample types that most frequently require testing and that are easily accessible if needed. Such a sample may be obtained by performing a biopsy from a living creature, or even from a dead/culled animal or from a deceased human. The method, location and type of sample taking are all parameters that a person skilled in the art can decide on in the knowledge of the given problem. Furthermore, as an especially preferable use of the present invention is in the case of livestock farming, the sample obviously especially preferably originates from livestock. Among livestock it is especially in the case of pigs, cattle, sheep, horses, oxen, goats, poultry, fish, turkeys, geese, pigeons, ducks, and ostriches where an infection can cause exceptionally great damage in an exceptionally short period of time due to the great density of animals, therefore the use of a fast and very precise identification method, such as the present invention, is especially important in the case of such livestock farming.

The comparison of the spectra recorded with the individual methods (such as step c) and step f)) and their evaluation (such as step d) and step g)) may be performed by visual inspection, for example. In the course of this an experienced person skilled in the art, for example by examining a spectrum recorded of the cell object, may determine which of the spectra available of known cell objects it is similar to. In the course of this process both spectra printed onto paper and spectra displayed on a computer screen can be used. In this case it may be necessary for the person performing the comparison to more precisely determine some of the measurement points on the spectra, which may take place by reading the scale on the spectrum, or, in the lack of this, with the help of a ruler or other measuring instrument. Nevertheless, today computer algorithms are widely and generally used for the comparison and evaluation of spectra. This is preferable from the point of view of the present invention, as the use of a computer algorithm gives a significantly faster and more precise result than visual evaluation; furthermore today it is an easily accessible and relatively cheap solution.

As mentioned above, the method according to the present invention is preferably implemented in a microfluidic device. The use of such a device is preferable because microfluidic devices are easy to transport because of the small size, therefore, for example, they can be easily transported to a livestock farm affected by an infection, by motorcar, for example, as a result of which the time required to identify the infection and determine the suitable antibiotic can be significantly reduced, as the samples do not have to be transported to a distant laboratory. Microfluidic devices are also especially suitable because the samples containing the cell objects can be provided in the amount, such as a small number of millilitres that can be handled by a microfluidic reactor.

The present invention also relates to a microfluidic device serving for implementing the method.

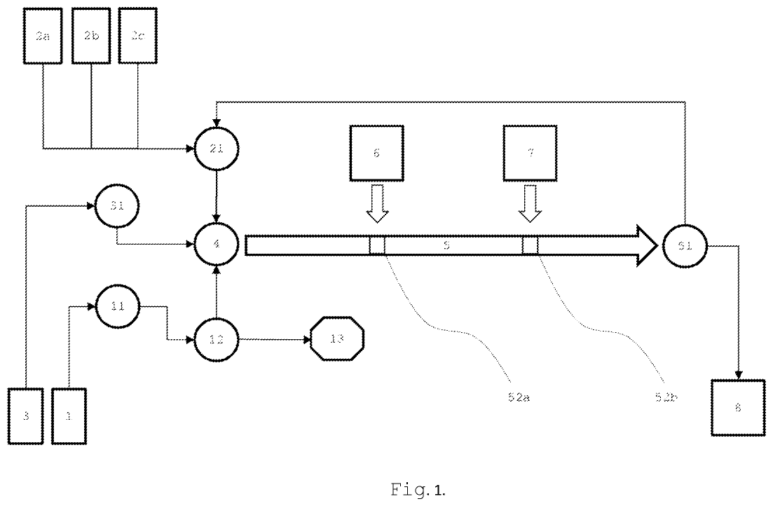

The operation scheme of the microfluidic device according to the invention is presented in FIG. 1. First of all it must be mentioned that the vessels used for storing liquids, the pumps used for moving the liquids, the liquid distribution valves, the drop dispensers, the microfluidic tubes and collectors used in microfluidic devices are all known according to the state of the art. It is also obvious for a person skilled in the art that the processes carried out in the microfluidic device, such as those carried out by the pumps, dispensers and other elements, are controlled and harmonised by a computer (microprocessor), which stores and executes the tasks on the basis of pre-inputted algorithms. For the sake of simplicity the control computer (microprocessor) is not depicted.

Therefore, the microfluidic device contains a sample holder 1, in which the prepared liquid sample is placed. The first pump 11 transports the liquid sample part from the sample holder 1 to the distribution valve 12. From here the liquid sample may progress along two paths: from the distribution valve 12 it either goes into the mass spectrometer 13 or to the drop dispenser 4. If the liquid sample goes to the mass spectrometer 13, its MS spectrum is recorded there and is stored in the control computer for later use. After the recording has been made the liquid sample part is taken to the collector 8 or another collector (not depicted).

The microfluidic device contains one or more test compound holders 2a, 2b, 2c. As an example three test compound holders have been depicted in the figure, however, it is obvious for a person skilled in the art that a microfluidic device according to the present invention may contain numerous holders and that the number of the holders does not influence the essence of the invention. The number of holders determines the number of different types of test compound that can be prepared for testing in the microfluidic device. If, for example, a microfluidic device is being designed for testing the five bacterial infections most frequently occurring on a poultry farm, then it is wise to install five test compound holders in the device for the five possible antibiotic solutions. If such an exemplary device does not have five holders, only fewer than this number, then it is only worth placing the antibiotic solution that is presumably effective for the identified bacteria after identification into the holder of the microfluidic device. The second pump 21 transports the solution(s) from the test compound holders to the drop dispenser 4. The solution of the test compounds is either measured on its own in order to be able to correct the spectra of the liquid sample parts containing the test compounds, or the solution of the test compounds is added to the liquid sample part presumably containing the cell object at one or more different concentrations in order to test the effect on the test compound.

The microfluidic device also contains an oil container 3, from which the third pump 31 transports the oil into the drop dispenser 4. The function of the oil in the oil container is to provide a medium that separates the individual liquid sample parts from each other in the liquid flowing in the microfluidic tube 5. In this respect the oil is viewed as the continuous phase. In the terms of the present invention this oil may be any apolar medium that does not substantially mix with water and so is able to perform the function of separator. It is also important that the oil does not contain substances that can exert an effect on the cell objects to be identified, as this would influence the test results. In addition to performing the separator function, the role of the oil is also to seal off the individual liquid sample parts from the air, which also makes it possible to perform testing of anaerobic cell objects. In the terms of the present invention the substances selected from the following group may be used as oil, without limitation: mineral oil, silicone oil, edible oil, vacuum oil, paraffin oil.

An important component of the microfluidic device is the drop dispenser 4. The function of this element is to dispense the liquids arriving from the individual holders and from other parts of the microfluidic device (see later on) through the operation of the pumps into the microfluidic tube 5 at the amount and in the order according to the commands of the control computer. In other words the drop dispenser 4 creates the series of liquid sample parts and oil flowing consecutively in the microfluidic tube 5.

The microfluidic tube 5 is a pipeline in which the liquid sample parts separated by the oil drops flow. The microfluidic tube 5 contains a first window 52a and a second window 52b physically identical or physically different to this. In other words depending on the architecture of the microfluidic device windows have to be used on the microfluidic tube 5 in the course of two steps, these two steps may be implemented at different, separated locations or at the same location, therefore an architecture is conceivable which does not contain two separate windows, but only one window, which is needed for the implementation of the two steps. Of these two steps the one is the destruction, which is carried out with the help of the destruction element 6, and the other is the measurement performed with the spectrophotometer 7.

According to the method according to the present invention the following takes place with the liquid sample part in the microfluidic device according to the present invention:
an MS spectrum is recorded of the liquid sample part using the mass spectrometer 13, which sample is then transported to a collector,
an other liquid sample part in the case of A) is subjected to destruction with the help of the destruction element 6, its spectrophotometry spectrum is recorded with the spectrophotometer 7, which sample is also transported to a collector,
the spectrophotometry spectrum of a further liquid sample part is recorded using the spectrophotometer 7 in the case of B), in a non-destructive way, without the addition of test compound, then
the spectrophotometry spectrum is recorded of this same sample using the spectrophotometer 7 but with the addition of test compound,
the test compound solution is added to this same liquid sample part and its spectrophotometry spectrum is measured as many times as necessary to reliably determine the effective concentration of the test compound, following this the liquid sample part is transported to a collector, and
the spectrum of the solution of the test compound is recorded.

The destruction element 6, then, affects the liquid sample part and is able to exert its destructive effect through the first window 52a. The window 52a is established in the way that depends on the type of destruction employed. In other words if it is necessary to add a material to perform the destruction, then this material may be added to the liquid sample through the window to the liquid sample passing by. If the destruction is performed using laser light, for example, then this window is established so that the laser light can penetrate here through the microfluidic tube 5 wall, in such a case the tube wall may be made from quartz. When non-destructive testing is performed on a liquid sample part, when such a liquid sample part passes by the destruction element is, naturally, inactive.

The liquid passing through the microfluidic tube 5 may be examined with the spectrophotometer 7 through the window 52b. Here the material of the microfluidic tube 5 is established so as to transmit the wavelength range used by the spectrophotometer. The spectrophotometer 7 may be, for example, a UV spectrophotometer or a Raman spectrometer.

With respect to that the intensity of the light emitted by spectrophotometers can be usually controlled, an architecture is conceivable with respect to the microfluidic device according to the invention in which the windows 52a and 52b are physically the same and it is through this single window that first radiating with light at a destructive intensity is carried out in order to implement the destruction step, then immediately after this the spectrum of the destructed sample may be recorded by using the spectrophotometer. In this preferable arrangement according to the invention the destruction element 6 and the spectrophotometer 7 are, obviously, the same device, which has two functions.

This is architecture, in other words when the two windows are physically the same window, may be typically implemented if the destruction element 6 is an electromagnetic radiation that gets to the liquid sample part through the same window that the spectrophotometry testing takes place thorough. The electromagnetic radiation is preferably a laser light emitted by the destruction element 6, even more preferably it is a laser light at a wavelength of approximately 530 nm. This is because the energy of laser light can be easily regulated, its beam can be easily handled and precisely directed. According to experience obtained incident laser light at a wavelength of approximately 530 nm is especially preferable for the destruction of cell objects.

According to an especially preferable embodiment the spectrophotometer 7 is a Raman spectrometer. This is because the Raman spectrometer, irrespective of the destruction step detailed above, it may be easily adapted to operation with a microfluidic device, both in terms of dimensions and its energy demand. Furthermore it is able to record spectra with a resolution that corresponds to the purposes of the present invention. Another advantage is that as Raman spectrometry testing does not damage the cell object, the same sample can be measured on several occasions, for instance when the effect of a concentration series of a test compound is to be tested.

According to another even more preferable embodiment the spectrophotometer 7 is a Raman spectrometer and the same Raman spectrometer serves as the destruction element 6. This is because the intensity of the laser light emitted by a Raman spectrometer can be controlled, as a result of which it is able to emit a laser light that destroys the cell objects, in this way then it is able to function as the destruction element 6, and is able to emit laser light with which the Raman spectrum of the cell object can be recorded without the cells being destroyed, in this way therefore, it is able to function as a spectrophotometer 7 for the non-destructive spectrophotometry step. In this case the first window 52a and the second window 52b form a single window.

According to another preferable embodiment of the invention an element that emits ultrasound may be used as the destruction element 6. It has been known to persons skilled in the art for a long time that ultrasound may be used to destroy cell objects. In this case the ultrasound emitting head protrudes into the liquid flowing in the microfluidic tube 5 through the first window 52a. Obviously in this case the first window 52a and the second window 52b are physically different.

The control computer of the microfluidic device is in possession of the information about where a given drop is moving in the microfluidic tube 5 at any given moment. In this way the computer is able to determine on the basis of the program inputted in advance what needs to be done with which liquid sample part, i.e. recording the spectrum with or without destruction, and whether test compound needs to be added, and if so what test compound at what concentration, etc.

The microfluidic tube 5 is connected to the second distribution valve 51, which either directs the liquid sample part so that it returns to the drop dispenser 4 via the second pump 21, or directs it so that it is taken to the collector 8.

If the liquid sample part is taken to the drop dispenser 4 through the second pump 51, then the second pump 21 mixes test compound solution from test compound holder 2a, 2b, or 2c with the liquid sample part. This liquid sample part with its concentration of test compound increased in this way is returned to the microfluidic tube 5 via the drop dispenser 4 and so it becomes possible to record the spectrophotometry spectrum with the spectrophotometer 7 of the same sample but with a greater concentration of test compound. Following this, through the distribution valve 51, this liquid sample part is either once again taken into the testing cycle or to the collector 8.

Those liquid elements are taken to the collector 8 that are no longer needed for the testing. The contents of the collector 8 may be disposed of in a way known of to a person skilled in the art with consideration to the cell objects, test compounds and other substances (such as oil) contained in the collector 8.

According to a preferable embodiment of the microfluidic device according to the invention its temperature may be controlled, most preferably in the temperature range of from approximately 37° C. to approximately 42° C. The reason for this is that different temperatures are optimal for different cell objects, therefore it is preferable to implement the method according to the invention at the temperature that is optimal for the tested cell object. For example in the case of infections the body temperature of various living creatures differs, and so it is worthwhile performing the test for the bacterium attacking the individual living creatures at this temperature, which is obvious for a person skilled in the art. For example it is worthwhile perform the tests in the case of chickens at 42° C. and at 37° C. in the case of a human infection. The ability to control temperature is also a preferable characteristic of the microfluidic device according to the invention because by maintaining the tested cell object at a given temperature it may multiply, in the case of which a more precise picture of the effect of the test compound may be obtained. For example in the case of the effect of antibiotics exerted on bacteria this is especially preferable because the effect of an antibiotic turns out very quickly by maintaining the optimal reproduction temperature of a bacterium. The reason for this is that the effect of an antibiotic consists of it preventing the building of a new cell wall when the bacterium reproduces, meaning that the new bacteria being formed suffer cell lysis, which can be easily identified using spectrophotometry.

An inert atmosphere may also be created in the microfluidic device according to the invention. This means that the sample does not come into contact with air in any of the steps, in other words the sample is not exposed to the effect of oxygen. Obviously for this the microfluidic device needs to be insulated (either the housing itself and/or the individual elements) and the introduction of the gas providing the inert atmosphere (e.g. nitrogen, argon) must also be ensured. All this belongs to the compulsory knowledge of a person skilled in the art. A testing environment with an inert atmosphere enables the testing of anaerobic cell objects, such as anaerobic bacteria.

### EXAMPLES

### Example 1

Examples Relating to MS Spectra

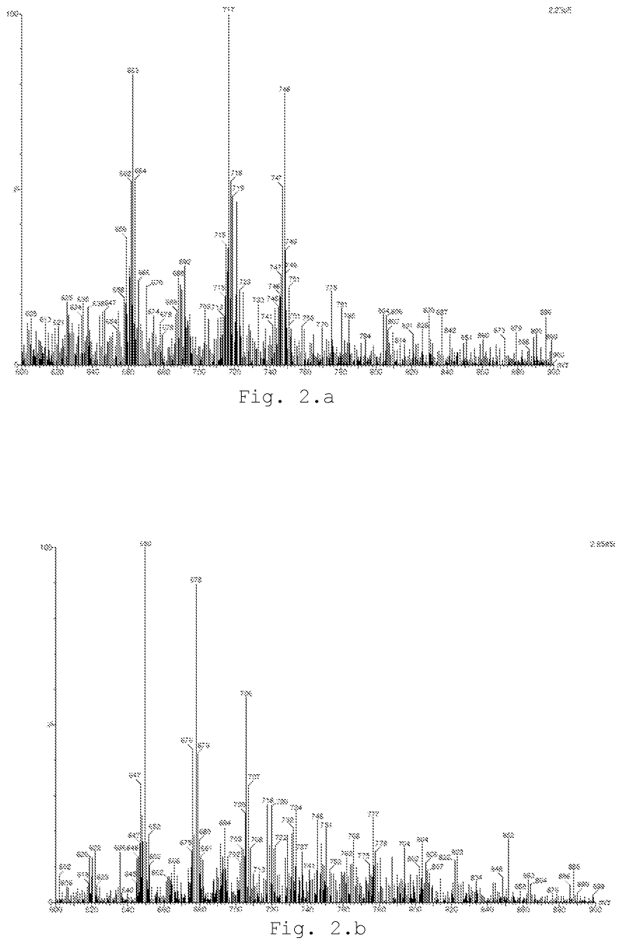

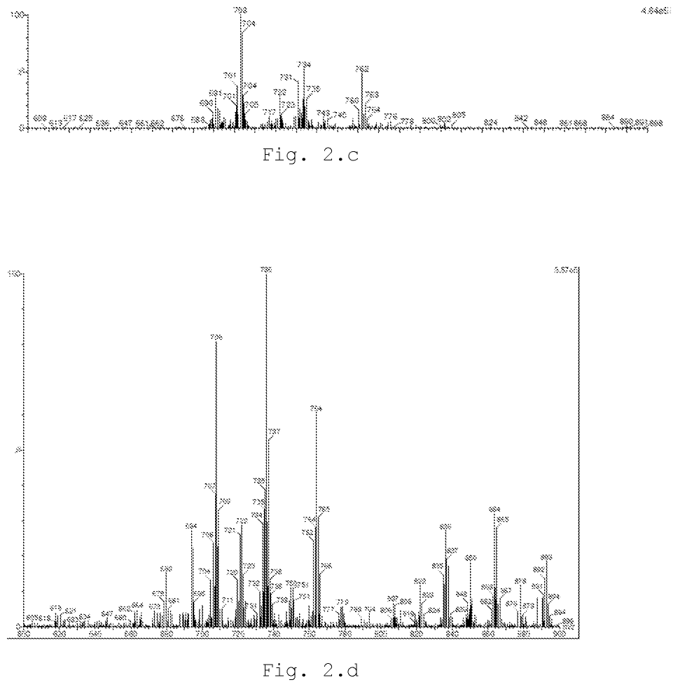

Figure series number 2 depicts the MS spectra of bacteria identified from samples taken from various places. FIG. 2.a shows the MS spectrum of Campylobacter jejuni in a sample originating from animal tissue, FIG. 2.b shows the MS spectrum of Clostridium perfringens in a sample originating from foodstuff, FIG. 2.c shows the MS spectrum of Escherichia coli in a sample originating from human tissue, and FIG. 2.d shows the MS spectrum of Staphylococcus auerus in a sample originating from animal tissue. The spectra were recorded in the 600 to 900 m/z range suitable for the detailed identification of phospholipids. It is can be clearly seen that the spectra have a great deal of detail, therefore are suitable for displaying the phospholipid fingerprint of individual bacteria.

### Example 2

Examples Relating to Raman Spectra

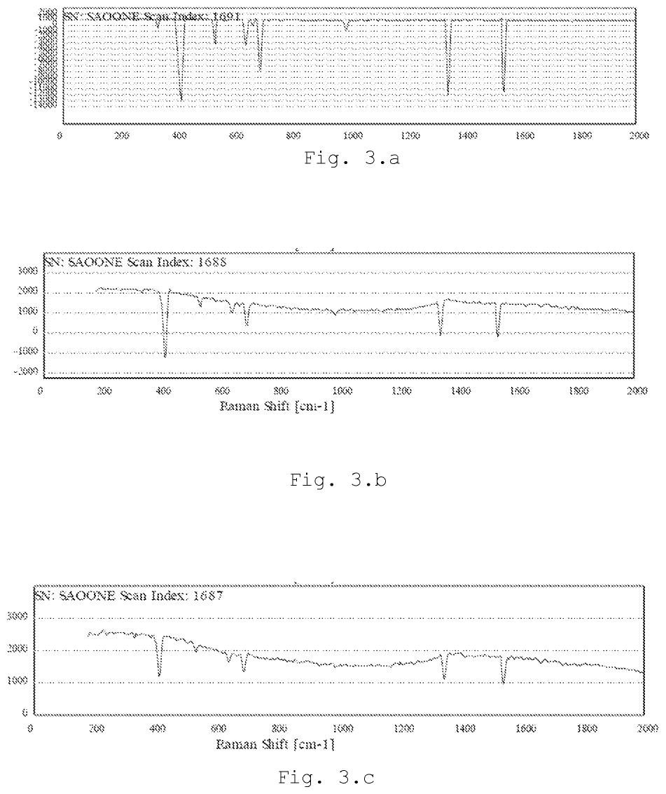

Figure series number 3 shows the Raman spectra of Escherichia coli, which in the terms of the present invention were recorded under non-destructive conditions. The spectrum according to FIG. 3.a was obtained with 785 nm wavelength laser light at 100% intensity, the spectrum according to FIG. 3.b was obtained with 785 nm wavelength laser light at 100% intensity following 20 minutes of sulphonamide antibiotic treatment, and the spectrum according to FIG. 3.c was obtained with 785 nm wavelength laser light at 100% intensity following 30 minutes of sulphonamide antibiotic treatment. It is easy to observe that the sulphonamide antibiotic treatment has significantly changed the Raman spectrum, in other words sulphonamide has an effect on Escherichia coli. It can also be observed that the peaks referring to the presence of cell objects have significantly diminished after 20 minutes of treatment, and these have diminished even more after 30 minutes of sulphonamide antibiotic treatment, in other words by using the non-destructive Raman spectroscopy test performed with 785 nm laser light the effect of sulphonamide antibiotic treatment on the Escherichia coli bacterium can be clearly demonstrated.

### Example 3

A mass spectrometry database was produced of the five bacteria most frequently infecting broiler chickens, namely the following: Escherichia coli, Staphylococcus auerus, Clostridium perfringens, Campylobacter jejuni, Salmonella. A total of 71 samples were tested originating from broiler chickens at a livestock farm. Among these the bacteria causing the infection was correctly identified in 68 cases by using the mass spectrometry method.

### Example 4

Based on experience a veterinary surgeon selected 10 broiler chickens which were suspected of being infected at a livestock farm. The vet performed autopsies of these in the morning hours, then took samples from the organs there were seen to be diseased, these samples were then tested using the traditional culturing method. These organs were subjected to mass spectroscopy testing for the five bacteria according to example 3. The testing of the 10 organ samples was performed in 3 hours, while an assessable result was obtained using the traditional procedure in 24 to 72 hours. The results were the same in 95% of the cases. The results are summarised in table 1.

TABLE 1
test on 10 broiler chicken

| | Infection | | | |
|---|---|---|---|---|
| | (determined | Infection | | |
| | using the | determined | | |
| | mass | using the | | |
| Sample | spectroscopy | traditional | | |
| name | method) | method | Remark | Score |
| Bone 1 | Coliform and | Coliform and | High | 1 |
| | Staphylococcus | Staphylococcus | concentration | |
| | | | Staphylococcus | |
| | | | and low | |
| | | | concentration | |
| | | | Staphylococcus | |
| Bone 2 | Coliform and | Coliform and | High | 1 |
| | Staphylococcus | Staphylococcus | concentration | |
| | | | Staphylococcus | |
| | | | and low | |
| | | | concentration | |
| | | | Staphylococcus | |
| Liver 1 | Coliform | Coliform | low | 1 |
| | | | concentration | |
| | | | infection | |
| Liver 2 | Campylobacter | Culture not | One week | 1 |
| | jejuni | successful | later the vet | |
| | | | confirmed the | |
| | | | Campylobacter | |
| | | | jejuni | |
| | | | infection | |
| Liver 3 | Coliform | Coliform | high | 1 |
| | | | concentration | |
| | | | infection | |
| Liver 4 | Coliform | Coliform | high | 1 |
| | | | concentration | |
| | | | infection | |
| Spleen 1 | Coliform and | Coliform | only slight | 0.5 |
| | Staphylococcus | | infection | |
| Spleen 2 | Coliform and | Coliform and | high | 1 |
| | Staphylococcus | Staphylococcus | concentration | |
| | | | infection | |
| Intestinal | Salmonella | Salmonella | Culture was | 1 |
| system | | | effective | |
| | | | after 52 | |
| | | | hours | |
| Follicle | Coliform | Coliform | high | 1 |
| (mature | | | concentration | |
| ovarian | | | infection | |
| cell, but | | | | |
| not yet | | | | |
| egg) | | | | |
| Total | | | | 9.5/10 |

Although in the present description the invention has been explained by mentioning bacteria as example for cell objects and antibiotics as test compounds, this does not mean that the invention is limited to these.

There are many non-bacterial cell objects for which various test compounds can be tested in a similar way. Basically any cell object may be analysed with the method and device according to the invention that can be put into a liquid sample to be tested in a microfluidic device and, from the other respect, the test compound to be tested causes a change in the cell object that may be identified using Raman spectroscopy. Examples of such a use are fungi-fungicide and alga-biocide active substances, which, naturally, also belong to the scope of protection of the present invention.

Among the advantages of the present invention it is important to emphasise that it can be used not only with a sample originating from a pure culture but also in the case of a sample taken directly from the tissue, contrary to the present practice where first of all pure cultures are always produced for the later tests, as the presence of cell objects not appearing in the spectra databases does not disturb identification. However, the presence of cell objects not occurring in the spectra databases can be identified with the device, as a phospholipid fingerprint that cannot be identified will be available. This is important information in a given case, namely this means that there is an unknown cell object in the sample, which may even be an infectious bacterium that was not expected. In this case the sample may be transported to a suitably equipped laboratory so that the unknown cell object can be identified using methods according to the state of the art. If it turns out that the cell object identified in this way is a bacterium that causes an infection, the spectrum relating to it can be recorded and loaded into the spectra databases of the microfluidic system according to the invention, and from this point onwards the identification of this to-date unknown cell object will no longer cause a problem. It should be noted that identification based on the use of a chromogenic culture substrate according to the state of the art is also a lengthy procedure, in addition to the process itself taking 24 to 72 hours, the development of a new chromogenic culture substrate for a strain of bacterium may take months or even years.

As detailed above the problem of multi-resistant bacteria is increasingly severe. The method and device according to the present invention is suitable for identifying one or more bacteria from a given sample and for determining the type and concentration of antibiotic effective against the identified bacterium. Therefore, if a bacterium present in the sample is resistant to the antibiotic that is effective against it according to the literature, by implementing the present method and using the present device, this turns out very quickly and so there is the opportunity to test several other, different antibiotics, and so in just a few hours the type and concentration of the antibiotic that is effective against the resistant or even multi-resistant bacterium can be identified in just a few hours. It should be noted here that calculating the effective dose for a given human or animal from the effective concentration determined according to the invention belongs to the compulsory knowledge of a person skilled in the art.

A further advantage of the present invention is that it enables identification faster than the state of the art. Usually 24 to 27 hours are required to implement the methods known according to the state of the art, with the present invention this time can be reduced to as little as 3 hours. The speed of identification is influenced, on the one part, by whether the available method can be used in a portable device so that the test can be performed on-site. As the method according to the invention can be adapted to a microfluidic device, the method complies with this requirement. As by installing the devices required to implement the present invention into a microfluidic device (including the aforementioned spectrophotometer, the mass spectrometer, etc.), a device will be available that can be easily transported by car. A MALDI-TOF, which, according to the state of the art, is suitable for determining the fingerprints of such microorganisms on the basis of the protein content of bacteria, is a device of such a size and is so expensive that from practical points of view excludes the adaptation of this approach into a portable device.

A further advantage of the invention is that if there are several cell objects in a sample which have their spectra in the databases, performing the simultaneous identification of these is not a problem. For example if infections occur at a livestock farm originating from two different bacteria, both of them can be identified using the method and device according to the invention and the type and dose of the antibiotic that is effective against both of them can also be determined.

The microfluidic device according to the invention is preferable for the implementation of the method according to the invention because this device also makes it possible to dilute the samples. In other words if during testing it turns out that an initial sample is too concentrated and an easily evaluable spectrum cannot be recorded from it, this problem can be solved by diluting the sample with a buffer solution.

Naturally, the microfluidic device may contain numerous buffer containers (not illustrated in FIG. 1) for this purpose, and the algorithm controlling the program or the operator may decide to dilute the affected liquid sample part.

It is obvious for a person skilled in the art that the method according to the present invention can be implemented in other devices apart from a microfluidic device, and can be simply adapted for pico-fluidic, nano-fluidic and meso-fluidic systems as well. In such cases the definition of cell object given in the present specification will be obviously amended.

1. Method for identifying test compounds that have an effect on cell objects, characterised by that the method comprises the following steps:
a) providing a liquid sample presumably containing a cell object,
b) performing mass spectrometry and/or destructive spectrophotometry testing of the liquid sample according to step a),
c) comparing the mass spectrometry spectrum and/or the spectrophotometry spectrum obtained in step b) with the elements of database(s) containing such spectra of known cell objects,
d) identifying the cell object present in the sample according to step a) in the course of the comparison according to step c),
e) the non-destructive spectrophotometry testing of the sample according to step a), in the course of which the non-destructive spectrophotometry spectrum of the sample is recorded in such a way that test compound is not added to it, and so that test compound is added to it at a given concentration or at several different concentrations, and recording of the spectrophotometric spectrum of the solution of the test compound,
f) comparing the spectrophotometric spectrum measured in the sample without the addition of any test compound and obtained in step e) with the spectrophotometry spectrum of one or more samples prepared with the addition of the test compound obtained in step e),
g) drawing a conclusion relating to the effective concentration of the test compound from the result of the comparison according to step f).

2. Method according to claim 1, characterised by that both the mass spectrometry and the destructive spectrophotometry test are performed in the course of step b) and the comparison according to step c) is performed with respect to both the mass spectrometry and the spectrophotometry spectra.

3. Method according to claim 1 or 2, characterised by that the mass spectrometry test according to step b) is performed in the 50 to 2000 mass to charge range, preferably in the 600 to 900 mass to charge range.

4. Method according to any of claims 1 to 3, characterised by that a UV spectrophotometry or Raman spectroscopy test is used as the spectrophotometry test during the implementation of step b) and/or step e), preferably a Raman spectroscopy test is used.

5. Method according to any of claims 1 to 4, characterised by that in the case of the destructive spectrophotometry test according to step b) the destruction is performed with ultrasound or electromagnetic radiation, preferably laser light, even more preferably using laser light at a wavelength of approximately 530 nm.

6. Method according to any of claims 1 to 5, characterised by that the cell object is a microorganism, preferably a bacterium, especially preferably a bacterium causing a human or animal disease, and the test compound in the case of this especially preferable form of implementation of the method is an antibiotic.

7. Method according to any of claims 1 to 6, characterised by that the elements of the databases according to step c) are spectra that were recorded using the same instrument as used when performing the tests according to step b).

8. Method according to any of claims 1 to 7, characterised by that the comparison according to step c) and/or the identification according to step d) and/or the comparison according to step f) and/or the drawing of the conclusion according to step g) are performed using a computer algorithm.

9. Method according to any of claims 1 to 8, characterised by that the sample is a piece of the tissue/part of an or human probably affected by an infection, a mucosal smear, a sample originating from body fluid, or a sample of epidermis.

10. Method according to claim 9, characterised by that the animal is a livestock animal, preferably pigs, cattle, sheep, horses, oxen, goats, poultry, fish, turkeys, geese, pigeons, ducks, ostriches.

11. Method according to any of claims 1 to 10, characterised by that it is implemented in a microfluidic device.

12. Device suitable for identifying test compounds having an effect on cell objects, characterised by that it is a microfluidic device and contains
a sample holder (1), a first pump (11), a first distribution valve (12), and a mass spectrometer (13),
one or more test compound holders (2a, 2b, 2c), and a second pump (21),
an oil container (3), and a third pump (31),
a drop dispenser (4), a microfluidic tube (5) containing a first window (52a) and a physically identical or physically different second window (52b), a destruction element (6), a spectrophotometer (7), a second distribution valve (51) and a collector (8)
wherein
a first pump (11) transports a part of the liquid sample from the sample holder (1) through a first distribution valve (12) into either the mass spectrometer (13) or a drop dispenser (4), and
a second pump (21) transports the given test compound solution from the one or more test compound holders (2a, 2b, 2c) into a drop dispenser (4),
a third pump (31) transports oil from the oil container (3) into the drop dispenser (4), and
the drop dispenser (4) dispenses a part of the liquid sample transported to it via the first pump (11) and the first distribution valve (12), to which it optionally mixes test compound solution transported to it via the second pump (21), and alternately the oil transported to it through the third pump (31) into the microfluidic tube (5) through which the liquid sample parts and the oil drops alternately flow, and where
in case A) the destruction element (6) exerts a destruction effect on the one or more liquid sample parts passing in front of the first window (52a), and the spectra of the one or more liquid sample parts passing in front of the second window (52b) are recorded using the spectrophotometer (7), and then the one or more liquid sample parts are transported to the collector (8) through the second distribution valve (51), or in case B) one or more liquid sample parts flowing from the drop dispenser (4) pass in front of the second window (52b) without destruction and the spectra of the one or more liquid sample parts are recorded with the spectrometer (7), which liquid sample parts are then transported to the collector (8) through the second distribution valve (51), or are returned into the drop dispenser (4) through the second pump (21), in the course of which test compound solution is added to the given liquid sample part from the appropriate test compound holder (2a, 2b, 2c) through the second pump (21) thereby increasing its test compound concentration, and in this way the changed liquid sample part once again passes in front of the second window (52b) through the microfluidic tube (5) without destruction and the spectrum of this liquid sample part is recorded with the spectrometer (7), then this is either transported into the collector (10), or in accordance with the former process the test compound concentration of the tested liquid sample part is increased even more, and the spectrum of the test compound transported from the appropriate test compound holder (2a, 2b, 2c) through the second pump (21) and the drop dispenser (4) into the microfluidic tube (5) is recorded with the spectrometer (7).

13. Device according to claim 12, characterised by that the first window (52a) and the second window (52b) of the microfluidic tube (5) are physically the same.

14. Device according to claim 12 or 13, characterised by that the spectrophotometer (7) is a UV spectrophotometer or a Raman spectrometer, preferably a Raman spectrometer.

15. Device according to any of claims 12 to 14, characterised by that the spectrometer (7) is a Raman spectrometer and this same Raman spectrometer also serves as the destruction element (6) as a result of the laser light emitted by it.

16. Device according to any of claims 12 to 14, characterised by that the destruction element (6) is an element emitting electromagnetic radiation, preferably an element emitting laser light, even more preferably an element emitting laser light at a wavelength of approximately 530 nm.

17. Device according to any of claims 12 to 14, characterised by that the destruction element (6) is an element emitting ultrasound.

18. Device according to any of claims 12 to 17, characterised by that its temperature may be controlled.

19. Device according to any of claims 12 to 18, characterised by that an inert atmosphere may be created in it.
